(19) United States
(12) Patent Application Publication
(10) Pub. No.: US 2017/0128227 A1
(43) Pub. Date: May 11, 2017

(54) CAGE DEVICE

(71) Applicant: MEDYSSEY CO., LTD., Jecheon-si (KR)

(72) Inventors: Dae-Seok Huh, Seoul (KR); Kyung-Min Chung, Seoul (KR); Youg Hai, Beijing (CN); Ho-Jung Kim, Namyangju-si (KR); Kwun-Mook Lim, Uijeongbu-si (KR)

(73) Assignee: MEDYSSEY CO., LTD., Jecheon-si (KR)

(21) Appl. No.: 15/346,348

(22) Filed: Nov. 8, 2016

(30) Foreign Application Priority Data
Nov. 9, 2015 (KR) 10-2015-0156360

(51) Int. Cl. A61F 2/44

(52) U.S. Cl. CPC A61F 2/447; A61F 2220/0016; A61F 2002/30011

(57) ABSTRACT

The present invention relates to a cage device inserted between adjacent vertebral bodies to be used for anterior vertebral body fusion. The cage device includes: a cage; and a staple fixing the cage between adjacent vertebral bodies, in which the staple is configured to include an upper fixed arm extending toward a back surface of the cage while being spaced apart from an upper surface of the cage, a lower fixed arm extending toward the back surface of the cage while being spaced apart from a lower surface of the cage, and a support part fixing the fixed arms to a front surface of the cage and ruggedness is formed on any one of upper and lower surfaces of the upper fixed arm 22 and the lower fixed arm 23.

### CROSS-REFERENCE TO RELATED APPLICATIONS

This application claims priority to Korean Patent Application No. 10-2015-0156360, filed on Nov. 9, 2015, which is incorporated herein by reference in its entirety.

### BACKGROUND OF THE INVENTION

Field of the Invention

Exemplary embodiments of the present invention relate to a cage device used for anterior vertebral body fusion.

Description of Related Art

A vertebral column consists of 32 to 35 vertebras forming a body and an intervertebral disk between the vertebras, that is, a disk and is a portion forming a backbone of our body that connects between a skull of an upper portion of a trunk and a pelvis of a lower portion thereof while forming a pillar of the trunk. The vertebra consists of 7 cervicals, 12 thoracics, 5 lumbers, 5 sacrums, and 3 to 5 coccyxes from above. In the case of an adult, 5 sacrums are fused to be one sacrum and 3 to 5 coccyxes are fused to be one coccyx.

When the vertebral column is damaged, the cage (artificial disk) is a device used to fuse bones by being inserted between adjacent vertebral bodies.

The existing fusion operation is performed by a method for removing a disc between adjacent vertebral bodies and inserting a cage (artificial disc) thereinto and then waiting for fusion thereof. However, since it takes at least six months to about two years to fuse the bone, the cage is separated for the period, and therefore complications, etc., are caused.

To solve the above problem, the method as illustrated in FIG. 1 is used. That is, a cage 1 is inserted and a plate 2 is fixed thereon to prevent the cage 1 from being separated until a bone is fused. The plate 1 is fixed by a screw 3.

However, the operation method gives a patient a great pain since an operation time is long and an incision site much larger than an operation range is required. Therefore, a device called “stand alone cage” in which the cage is coupled with the screw as illustrated in FIG. 2 has been developed. However, there is a problem in that the product requires a hole that can fix the screw 3 and thus a front portion is thicker than necessary and a locking mechanism for preventing the screw 3 from being separated needs to be additionally installed.

Further, the vertebral bodies slightly differs for each person, and therefore when the cage is inserted, the cage needs to be inserted deeper or less on person-to-person basis. However, the “stand alone cage” is attached with the plate, and therefore cannot perform the foregoing control.

### RELATED ART DOCUMENT

### Patent Document

Korean Patent Laid-Open Publication No. 10-2011-0011049

### SUMMARY OF THE INVENTION

An embodiment of the present invention is directed to a cage device capable of performing an operation by incising only a relatively narrower portion when performing an insertion operation of a cage (artificial disc) between adjacent vertebral bodies and greatly shortening an operation time by simplifying an insertion method, and firmly fixing the cage between the vertebral bodies after the operation.

Another embodiment of the present invention is directed to a cage device in which a screw is not used, and therefore a separate locking mechanism is unnecessary and an operation method is also simple.

Still another embodiment of the present invention is directed to a cage device capable of controlling an insertion depth of the cage to be different on patient-to-patient.

Other objects and advantages of the present invention can be understood by the following description, and become apparent with reference to the embodiments of the present invention. Also, it is obvious to those skilled in the art to which the present invention pertains that the objects and advantages of the present invention can be realized by the means as claimed and combinations thereof.

In accordance with an embodiment of the present invention, there is provided a cage device inserted between adjacent vertebral bodies to be used for anterior vertebral body fusion, the cage device including: a cage; and a staple fixing the cage between adjacent vertebral bodies, in which the staple is configured to include an upper fixed arm extending toward a back surface of the cage while being spaced apart from an upper surface of the cage, a lower fixed arm extending toward the back surface of the cage while being spaced apart from a lower surface of the cage, and a support part fixing the fixed arms to a front surface of the cage and ruggedness is formed on any one of upper and lower surfaces of the upper fixed arm 22 and the lower fixed arm 23.

### BRIEF DESCRIPTION OF THE DRAWINGS

FIG. 1 is a diagram illustrating the existing method for fixing a cage between adjacent vertebral bodies.

FIG. 2 is a diagram illustrating the structure of the existing stand alone cage developed to prevent a cage from being separated between vertebral bodies.

FIGS. 3 to 6 are diagrams illustrating a structure of a cage device according to an embodiment of the present invention.

FIG. 7 is a diagram illustrating a form in which the cage device according to the embodiment of the present invention is inserted between adjacent vertebral bodies by an operation.

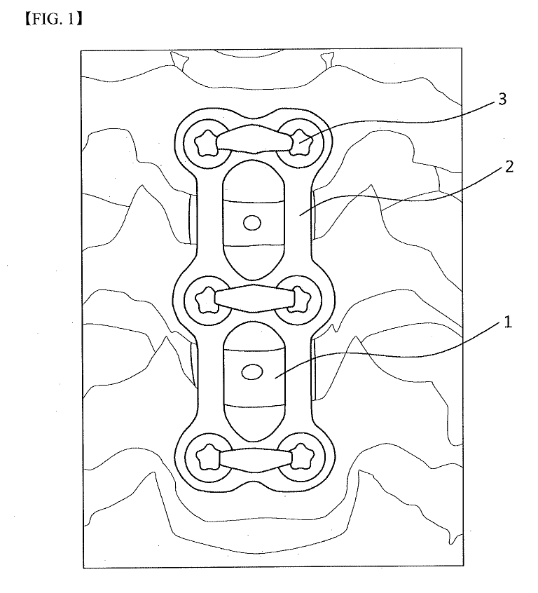

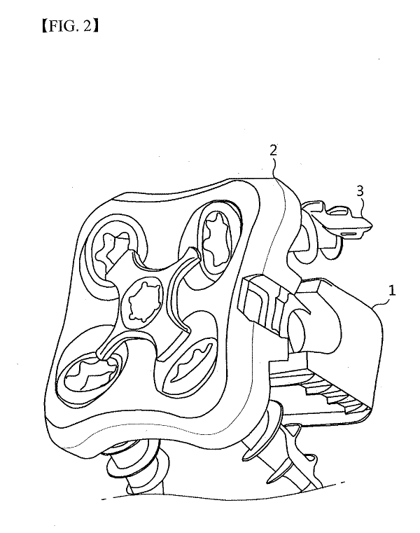

### DESCRIPTION OF SPECIFIC EMBODIMENTS

Hereinafter, exemplary embodiments of the present invention will be described in detail with reference to the accompanying drawings. Further, when it is determined that the detailed description of the known art related to the present invention may obscure the gist of the present invention, the detailed description thereof will be omitted.

The following description and drawings illustrate specific embodiments to easily perform an apparatus and a method described by those skilled in the art. Another embodiment may include structurally and logically different modifications. Unless clearly requested, individual components and functions may be generally selected and an order thereof may be changed. Portions and features of several embodiments may be included in other embodiments or may be replaced by other embodiments.

The present invention relates to a cage device 100 inserted between adjacent vertebral bodies to be used for anterior vertebral body fusion.

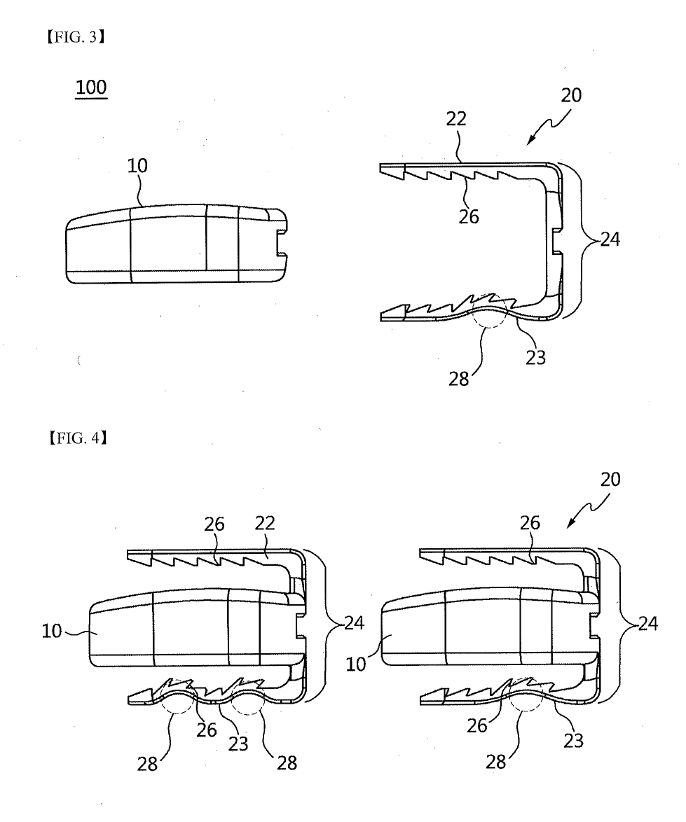

As illustrated in FIG. 3, the cage device 100 includes a cage 10 and a staple 20 fixing the cage between adjacent vertebral bodies.

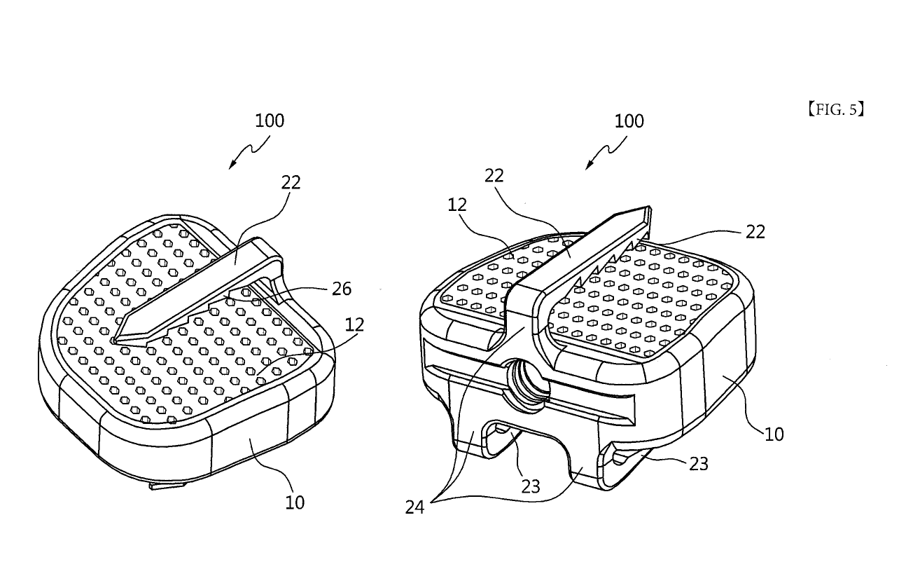

As illustrated in FIG. 4, the staple 20 is configured to include an upper fixed arm 22 extending toward a back surface of the cage while being spaced apart from an upper surface of the cage 10, a lower fixed arm 23 extending toward the back surface of the cage while being spaced apart from a lower surface of the cage, and a support part 24 fixing the fixed arms to a front surface of the cage.

The present invention relates to the cage device in which ruggedness 26 is formed on any one of upper and lower surfaces of the upper fixed arm 22 and the lower fixed arm 23.

In the cage device 100, the ruggedness 26 may be more preferably formed on the lower surface of the upper fixed arm 22 and the upper surface of the lower fixed arm 23, that is, a side facing the cage 10.

In the cage device 100 according to the embodiment of the present invention, as illustrated in FIG. 5, the support part 24 of the staple may be integrally formed with the cage 10 or formed independent of the cage 10 to be fixed to the cage 10 by a general fixing means.

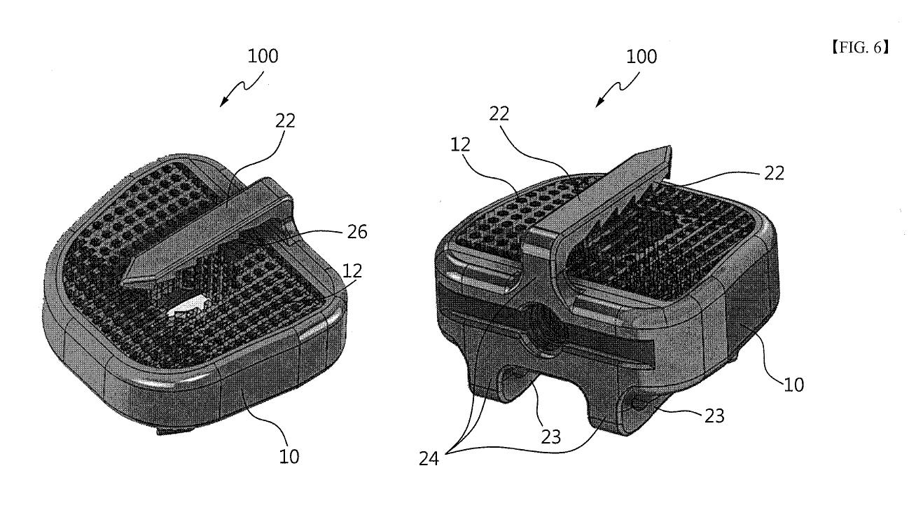

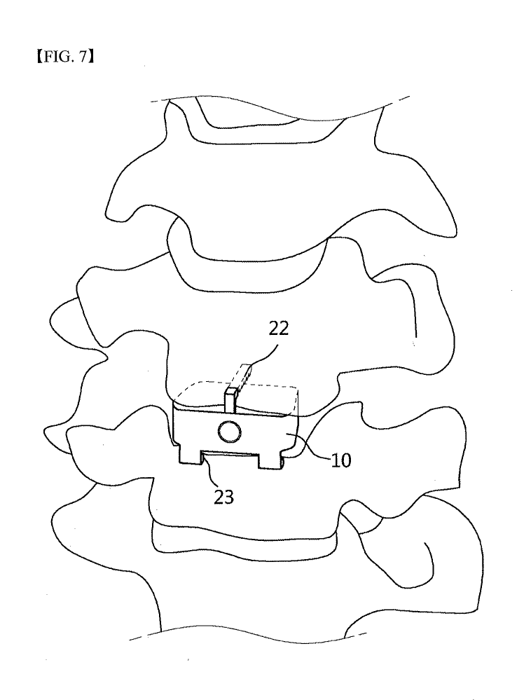

In particular, when the support part 24 of the staple is integrally formed with the cage 10, the support part 24 may be manufactured at a time in a form as illustrated in FIG. 6 by using a 3D printer, or the like, thereby simplifying a manufacturing process and firming a coupling.

Further, in the cage device 100 according to the embodiment of the present invention, as illustrated in FIG. 4, at least one of the upper fixed arm 22 and the lower fixed arm 23 includes more than one bent part 28 bent up and down.

The bent part 28 greatly improves a fixing force for the vertebral bodies of the fixed arm, thereby effectively preventing the cage 10 from being separated between adjacent vertebral bodies.

Further, in the cage device 100 according to the embodiment of the present invention, as illustrated in FIGS. 5 and 6, any one of the upper fixed arm 22 and the lower fixed arm 23 is configured to include more than two fixed arms.

As described above, when any one of the upper fixed arm 22 and the lower fixed arm 23 is configured of more than two fixed arms, as illustrated in FIGS. 5 and 6, the support part 24 fixing more than two fixed arms to the cage 100 may be formed in the form in which each of the fixed arms are independently fixed to the cage 10.

Further, the support part 24 may also be formed in the form in which each of the fixed arms is together coupled with the support part. In this case, the support part 24 is not manufactured in the form in which each of the fixed arms are independently fixed to the cage 10, for example, the support part 24 is formed in a squared shape and more than two fixed arms may also be fixed to one side (not illustrated).

In the cage device 100 according to the embodiment of the present invention, the ruggedness formed at the fixed arms may be formed in a sawtooth 26 shape as illustrated in FIG. 4.

Further, the ruggedness may also be formed in a general form used in the art. That is, as long as the ruggedness has a form performing a function of preventing the cage 10 from being separated between adjacent vertebral bodies, the shape of the ruggedness is not specially limited. The ruggedness may also be formed in a spike shape.

In the cage device 100 according to the embodiment of the present invention, the form of the cage 10 is not specially limited and all the forms generally used in the art may be adopted.

Preferably, as illustrated in FIG. 6, the form in which a corner of the cage is rounded and a width of the back surface of the cage is smaller than that of the front surface thereof may be preferable in insertion easiness, fusion efficiency, etc., at the time of the operation. In particular, manufacturing the upper and lower surfaces of the cage 10 to include a porous structure 12 is preferable in the fusion efficiency.

FIG. 7 is a diagram illustrating a form in which the cage device according to the embodiment of the present invention is inserted between adjacent vertebral bodies by an operation. As can be appreciated in the drawings, the cage device according to the embodiment of the present invention may be mounted by a method for preparing holes through which the upper fixed arm 22 and the lower fixed arm 23 of the staple may be inserted into vertebral bodies over and under a portion into which the cage 10 will be inserted and inserting the upper fixed arm 22 and the lower fixed arm 23 of the staple into the holes prepared on the vertebral bodies while inserting the cage 10 between adjacent vertebral bodies.

As described above, the cage device according to the embodiment of the present invention has the simple structure but provides the very excellent fixed performance of the cage. Therefore, it is possible to perform an operation by incising only the narrow portion, greatly shorten the operation time by simplifying the insertion method, and firmly fix the cage between adjacent vertebral bodies after the operation (which will be again described below), at the time of the insertion operation of the cage.

Further, according to the embodiment of the present invention, the cage device may control the insertion depth of the cage to be different on patient-to-patient basis, thereby facilitating the operation and performing the customized operation depending on the patient's state.

According to the embodiments of the present invention, the cage device may have the simple structure but have the very excellent fixed performance of the cage. Therefore, it is possible to perform an operation by incising only the relatively narrow portion, greatly shorten the operation time by simplifying the insertion method, and firmly fix the cage between adjacent vertebral bodies after the operation, at the time of the insertion operation of the cage.

In addition, according to the embodiments of the present invention, the cage device does not use the screw, thereby making the separate locking mechanism unnecessary and simplifying the operation method.

Further, according to the embodiment of the present invention, the cage device may control the insertion depth of the cage to be different on patient-to-patient basis, thereby facilitating the operation and performing the customized operation depending on the patient's state.

Although the present invention is described with reference to the preferred embodiments described above, the present invention may be variously modified or changed without departing from the subject matter and scope of the present invention. Therefore, the appending claims will include the modifications or changes as long as they belong to the subject matter of the present invention.

What is claimed is:

1. A cage device inserted between adjacent vertebral bodies to be used for anterior vertebral body fusion, comprising:
a cage; and
a staple fixing the cage between adjacent vertebral bodies,
wherein the staple is configured to include an upper fixed arm extending toward a back surface of the cage while being spaced apart from an upper surface of the cage, a lower fixed arm extending toward the back surface of the cage while being spaced apart from a lower surface of the cage, and a support part fixing the fixed arms to a front surface of the cage, and
ruggedness is formed on any one of upper and lower surfaces of the upper fixed arm and the lower fixed arm.

2. The cage device of claim 1, wherein the ruggedness is formed on the lower surface of the upper fixed arm and the upper surface of the lower fixed arm.

3. The cage device of claim 1, wherein the support part of the staple is integrally formed with the cage or is formed independent of the cage to be fixed to the cage.

4. The cage device of claim 1, wherein the support part of the staple is integrally formed with the cage.

5. The cage device of claim 1, wherein at least one of the upper fixed arm and the lower fixed arm includes more than one bent part bent up and down.

6. The cage device of claim 1, wherein at least any one of the upper fixed arm and the lower fixed arm consists of more than two fixed arms.

7. The cage device of claim 6, wherein when any one of the upper fixed arm and the lower fixed arm consists of more than two fixed arms, the support part fixing more than two fixed arms to the cage is formed in a form in which each of the fixed arms is independently fixed to the cage or each of the fixed arms is together coupled with the support part.

8. The cage device of claim 1, wherein the ruggedness is formed in a sawtooth shape.

9. The cage device of claim 1, wherein the upper and lower surfaces of the cage have a porous structure.
